(19) United States
(12) Patent Application Publication
(10) Pub. No.: US 2021/0353811 A1
(43) Pub. Date: Nov. 18, 2021

(54) MANAGEMENT APPARATUS, MANAGEMENT SYSTEM, MANAGEMENT METHOD, AND PROGRAM FOR MANAGING RECORDS OF PROCESSES USING TEST PAPER HAVING DISCOLORATION AREA

(71) Applicant: Canon Kabushiki Kaisha, Tokyo (JP)

(72) Inventors: Motohiro Furusawa, Shizuoka (JP); Ichiro Yasumaru, Shizuoka (JP)

(21) Appl. No.: 17/290,276

(22) PCT Filed: Nov. 13, 2019
(86) PCT No.: PCT/JP2019/044547

§ 371 Date: Apr. 30, 2021

(30) Foreign Application Priority Data
Nov. 28, 2018 (JP) 2018-222689

(51) Int. Cl. A61L 2/28; G16H 40/20

(52) U.S. Cl. CPC A61L 2/28; A61L 2202/24; A61L 2202/14; G16H 40/20

(57) ABSTRACT

A management apparatus that manages a record of a predetermined process carried out using test paper, which has identification information and a discoloration area, is provided. The apparatus includes: obtaining means for obtaining the identification information and a measurement result for the discoloration area, from a measurement apparatus; and recording means for determining a field to be recorded in a target record based on already-recorded fields in the target record which corresponds to the identification information, and recording information in the determined field. The record includes a first field in which the achievement degree of the predetermined process is recorded, and one or more second fields in which information is recorded earlier than a timing at which the achievement degree of the predetermined process is recorded in the first field.

### TECHNICAL FIELD

The present invention relates to a technique for managing records of a predetermined process carried out using test paper which has a discoloration area that changes color depending on the achievement degree of the predetermined process.

### BACKGROUND ART

Sterilization targets, such as medical tools used at hospitals, are subjected to sterilization processes. At this time, what is known as a chemical indicator (“CI” hereinafter) for sterilization is used in order to determine the achievement degree of the sterilization process on the sterilization targets. A CI has a discoloration area that changes color in accordance with the achievement degree of the conditions required for the sterilization process, which uses a sterilizing agent (vapor, hydrogen peroxide, or the like). Japanese Patent No. 3414291 discloses a management apparatus that manages sterilization process records. According to Japanese Patent No. 3414291, the management apparatus includes CI determination results in the sterilization process records.

For example, the achievement degree of the sterilization process cannot be accurately determined if a CI which has an initial problem with the color in the discoloration area is used. In other words, to improve the quality of the sterilization process, it is necessary to prevent CIs having initial problems from being used. Although Japanese Patent No. 3414291 discloses including the CI determination result in the record of the sterilization process, it does not disclose a configuration for preventing the use of a CI having an initial problem. Furthermore, in order to verify the details of the work involved in the sterilization process after the fact and improve the quality of the sterilization process, it is necessary to record various types of related information, such as the sterilization target and the sterilization processing device used in the sterilization process, along with the result of the sterilization process. However, when there are many other tasks to complete, including using the sterilization target immediately after the sterilization process, human error may arise, such as the operator who handles the sterilization process forgetting to record this related information. Such human error must be prevented in order to improve the quality of the sterilization process on the basis of the sterilization process records.

### SUMMARY OF INVENTION

According to an aspect of the present invention, a management apparatus that manages a record of a predetermined process carried out using test paper, the test paper having identification information and a discoloration area, a color of the discoloration area changing in accordance with an achievement degree of the predetermined process is provided. The apparatus includes: obtaining means for obtaining the identification information and a measurement result for the discoloration area, from a measurement apparatus that measures the discoloration area of the test paper and reads the identification information; and recording means for determining a field to be recorded in a target record based on already-recorded fields in the target record which, among records recording the predetermined process, corresponds to the identification information obtained by the obtaining means, and recording information in the determined field to be recorded. The record includes a first field in which the achievement degree of the predetermined process is recorded, and one or more second fields in which information is recorded earlier than a timing at which the achievement degree of the predetermined process is recorded in the first field.

Further features of the present invention will become apparent from the following description of exemplary embodiments with reference to the attached drawings.

### BRIEF DESCRIPTION OF DRAWINGS

FIG. 1 is a diagram illustrating the configuration of a management system including a management apparatus according to one embodiment.

FIG. 2 is a diagram illustrating the configuration of a measurement apparatus according to one embodiment.

FIG. 3 is a diagram illustrating a CI according to one embodiment.

FIGS. 4A to 4C are diagrams illustrating a management information table according to one embodiment.

FIG. 5 is a flowchart illustrating a process for recording to a management information table according to one embodiment,

FIG. 6 is a flowchart illustrating a process for recording to a management information table according to one embodiment.

FIG. 7 is a diagram illustrating a management information table according to one embodiment.

FIG. 8 is a flowchart illustrating a process for recording to a management information table according to one embodiment.

FIG. 9 is a diagram illustrating a management information table according to one embodiment.

FIG. 10 is a flowchart illustrating a process for recording to a management information table according to one embodiment.

FIG. 11 is a diagram illustrating a management information table according to one embodiment.

FIG. 12 is a function block diagram illustrating functions realized by a PC according to one embodiment.

### DESCRIPTION OF EMBODIMENTS

Hereinafter, exemplary embodiments of the present invention will be described with reference to the drawings. Note that the following embodiments are to be taken as examples only, and the present invention is not intended to be limited by the embodiments. Note also that constituent elements not necessary for the descriptions of the embodiments have been omitted from the drawings.

### First Embodiment

FIG. 1 is a diagram illustrating a management system including a management apparatus 100 of sterilization processes according to the present embodiment. The management apparatus 100 includes a personal computer (PC) 15 and a measurement apparatus 1. The management apparatus 100 is connected to a network, and can communicate with a server apparatus 14 over the network. Although FIG. 1 illustrates two management apparatuses 100, any desired number of management apparatuses 100 can be used. When a plurality of management apparatuses 100 are used, the plurality of management apparatuses 100 are placed in different locations. For example, if the management system illustrated in FIG. 1 is inside a hospital, a management apparatus 100 is placed in each location where some of the work involved in the sterilization process is carried out, such as the room where the sterilization processing apparatus is placed to carry out the sterilization process, the room where the sterilization targets are used, and so on. The server apparatus 14 holds a management information table which has records for individual sterilization processes. The management apparatus 100 can add records to the management information table held by the server apparatus 14, and can furthermore add information to the fields in the records. More specifically, the management apparatus 100 can obtain information recorded in the management information table held by the server apparatus 14, which is an external apparatus, over the network. Furthermore, the management apparatus 100 can communicate information to be recorded in the management information table to the server apparatus 14 over the network, and can cause the server apparatus 14 to record that information in the management information table.

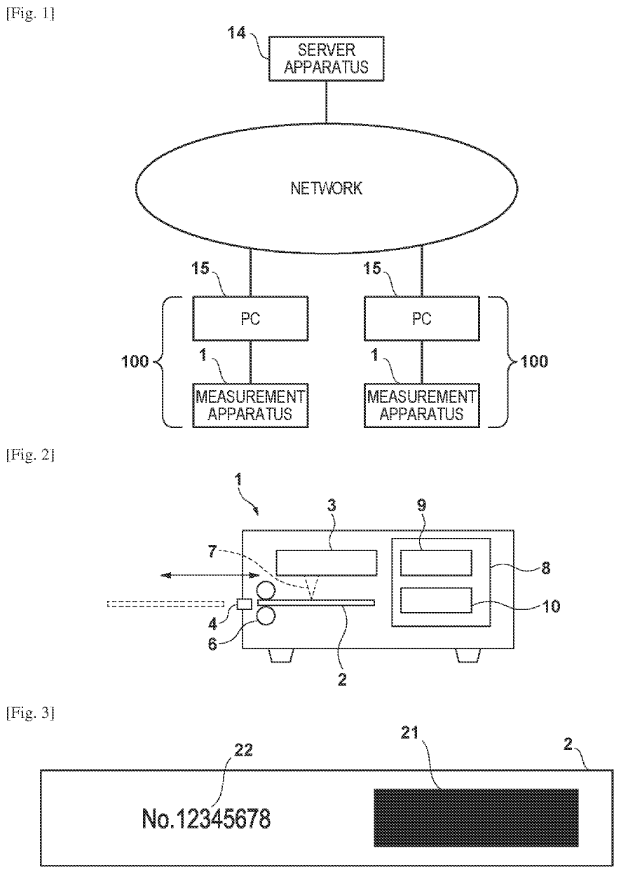

FIG. 2 is a cross-sectional view of the measurement apparatus 1. The measurement apparatus 1 includes an insertion part 4 for a CI 2, and a conveyance roller pair 6 that conveys the CI 2, which has been inserted into the insertion part 4, to a measurement position on the downstream side. When an operator (measurer) inserts the CI 2 from the insertion part 4 as indicated by the dotted line in FIG. 2, the conveyance roller pair 6 conveys the CI 2 to the measurement position, which is indicated by the solid line. FIG. 3 is a diagram illustrating the CI 2 according to the present embodiment. The CI 2 is a sheet-shaped test paper, and has on its surface a discoloration area 21 which has been subjected to a chemical process. A color of the discoloration area 21 changes in accordance with the achievement degree of the sterilization process. For example, the discoloration area 21 of the CI 2 is configured to start with a color value C#1 in an initial state, change to color values C#2 to C#9 in accordance with the achievement degree of the sterilization process, and ultimately stabilize at a color value C#10. The CI 2 also has an identification part 22, which includes identification information for classifying and identifying individual CIs 2. In FIG. 3, the identification part 22 is the serial number of the CI 2, printed on the base of the CI 2. However, the identification part 22 can be a printed barcode as well. Alternatively, the identification part 22 can be an IC tag (radio tag) affixed to the CI 2.

Returning to FIG. 2, a measurement unit 3 is arranged on the side opposite from the measurement position. The measurement unit 3 uses light 7 to read an optical measurement value pertaining to the color value of the discoloration area 21 on the CI 2, e.g., a spectral reflectance. If the identification part 22 is a serial number or a barcode printed on the CI 2, the measurement unit 3 optically reads the identification part 22 on the CI 2 as well. Note that if the identification part 22 is an IC tag, the measurement unit 3 communicates with the IC tag and reads the identification information stored in the IC tag. Thus the measurement unit 3 not only measures the optical measurement value of the discoloration area 21, but also functions as a reading unit which reads the identification part 22. After the CI 2 is measured by the measurement unit 3, the CI 2 is discharged from the measurement apparatus 1 by rotating the conveyance roller pair 6 in reverse. A control unit 8 controls the measurement apparatus 1. Various types of information used for measurement are stored in a storage unit 9 of the control unit 8. A computation processing unit 10 of the control unit 8 determines the color value of the discoloration area 21 of the CI 2 on the basis of the result of the measurement by the measurement unit 3, e.g., the spectral reflectance, and then outputs color information indicating the determined color value along with the read identification information of the CI 2 to the PC 15.

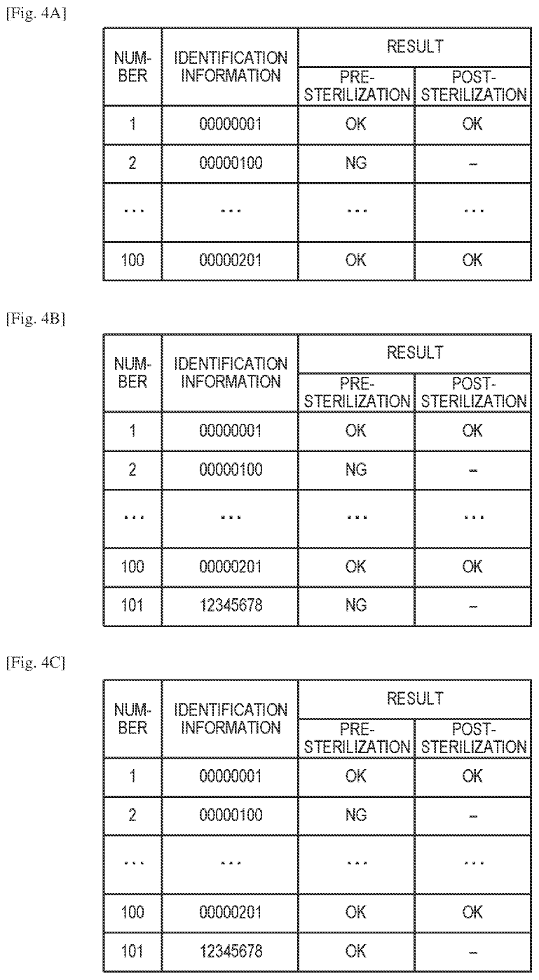

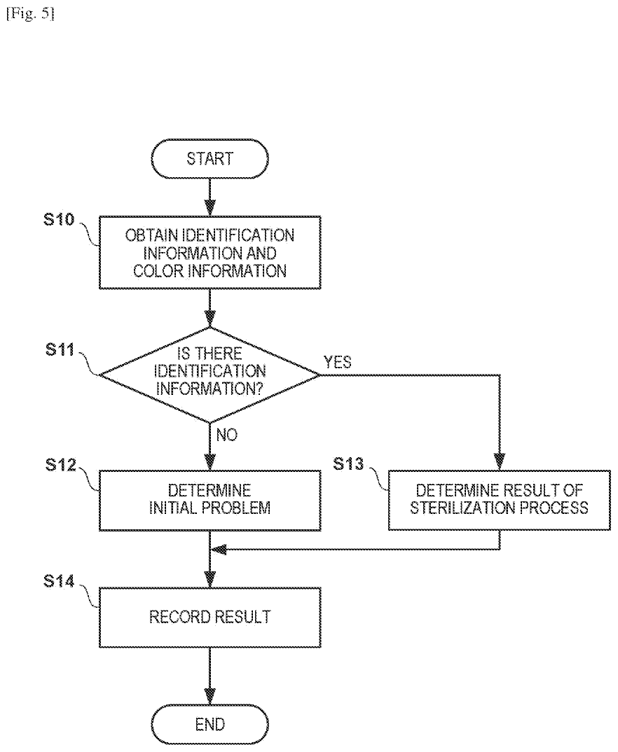

FIG. 4A illustrates the management information table held by the server apparatus 14 according to the present embodiment. The “number” field indicates the number of each record. A single record is provided for each piece of CI 2 identification information, i.e., for each individual piece of test paper. The “identification information” field holds the identification information of the CI 2. In the present embodiment, the “result” field has two subfields, i.e., “pre-sterilization” and “post-sterilization”. In the present embodiment, the CI 2 is measured by the measurement apparatus 1 both after and sterilization process and before the sterilization process. Whether or not the CI 2 has an initial problem is determined on the basis of the result of the measurement of the CI 2 by the measurement apparatus 1 before the sterilization process, and the determination result is held in the pre-sterilization subfield. Additionally, whether the sterilization process has reached a predetermined achievement degree is determined on the basis of the result of the measurement apparatus 1 measuring the CI 2 after being used in the sterilization process, and the determination result is held in the post-sterilization subfield.

For example, when the management information table has the content illustrated in FIG. 4A, the CI 2 having the identification information of “12345678”, indicated in FIG. 3, is read by the measurement apparatus 1. A color value Ct resulting from the measurement (called a “measured color value” hereinafter) and the identification information are then output to the PC 15. The PC 15 determines whether or not a record for recording which corresponds to the identification information “12345678” is present in the management information table illustrated in FIG. 4A. Here, assume that a record for recording which corresponds to the identification information “12345678” is not present in the management information table. In this case, the PC 15 adds a new record (number 101) to the management information table as a target record. Then, the identification information “12345678” is recorded in the identification information field of the target record, as indicated in FIGS. 4B and 4C. Because the target record was not present, the PC 15 determines that the measurement result obtained from the measurement apparatus 1 is from before the sterilization process was carried out. In this case, the PC 15 determines whether or not a color difference between the color value C#1, which indicates the initial color of the discoloration area 21 of the CI 2, and the measured color value Ct, is greater than a threshold. If the color difference between the color value C#1 and the measured color value Ct exceeds the threshold, “NG” is recorded in the pre-sterilization subfield, as indicated in FIG. 4B. However, if the color difference between the color value C#1 and the measured color value Ct does not exceed the threshold, “OK” is recorded in the pre-sterilization subfield, as indicated in FIG. 4C. The PC 15 displays whether or not the color difference between the color value C#1 and the measured color value Ct exceeds the threshold in a display unit such as a monitor. Furthermore, if the color difference between the color value C#1 and the measured color value Ct of the CI 2 exceeds the threshold, an indication that the CI 2 cannot be used to determine the achievement degree of the sterilization process can be displayed in the display unit.

Furthermore, assume that when the content of the management information table is as illustrated in FIG. 4C, the measurement apparatus 1 has measured the CI 2 having the identification information “12345678”, and has output the measured color value Ct and the identification information “12345678” to the PC 15. In this case, a target record corresponding to the identification information “12345678” is present in the management information table indicated in FIG. 4C, and thus the PC 15 can determine that the measurement result obtained from the measurement apparatus 1 is from after the sterilization process was carried out. In this case, the PC 15 determines the color, among the plurality of color values C#1 to C#10 which the discoloration area 21 can take on, that has the lowest color difference relative to the measured color value Ct. If, for example, the value having the smallest color difference relative to the measured color value Ct is any one of the color values C#8 to C#10, a determination of “OK” is made, whereas if such is not the case, a determination of “NG” is made. The PC 15 displays the determination result for a user, and records the determination result in the post-sterilization subfield of the target record.

FIG. 5 is a flowchart illustrating processing carried out by the PC 15 according to the present embodiment. In step S10, the PC 15 obtains the identification information and color information (information indicating the measured color value Ct) from the measurement apparatus 1. Then, in step S11, the PC 15 determines whether or not a target record, in which that identification information is recorded, is present in the management information table. If a target record in which that identification information is recorded is not present in the management information table, in step S12, the PC 15 determines whether there is an initial problem. Specifically, the PC 15 determines whether the CI 2 corresponds to “OK” or “NG” by comparing the color difference between the measured color value Ct and the color value C#1, which is the initial color, with the threshold. Then, in step S14, the PC 15 adds a target record, in which the identification information obtained in step S10 is recorded in the identification information field, and records the determination result in the pre-sterilization subfield. On the other hand, if in step S11 there is a target record, in which the corresponding identification information is recorded, present in the management information table, the PC 15 determines the result of the sterilization process in step S13. Specifically, the PC 15 determines whether the sterilization process corresponds to “OK” or “NG” by comparing the color value C#1 to color value C#10 with the measured color value Ct. The determination result is recorded in the post-sterilization subfield in step S14.

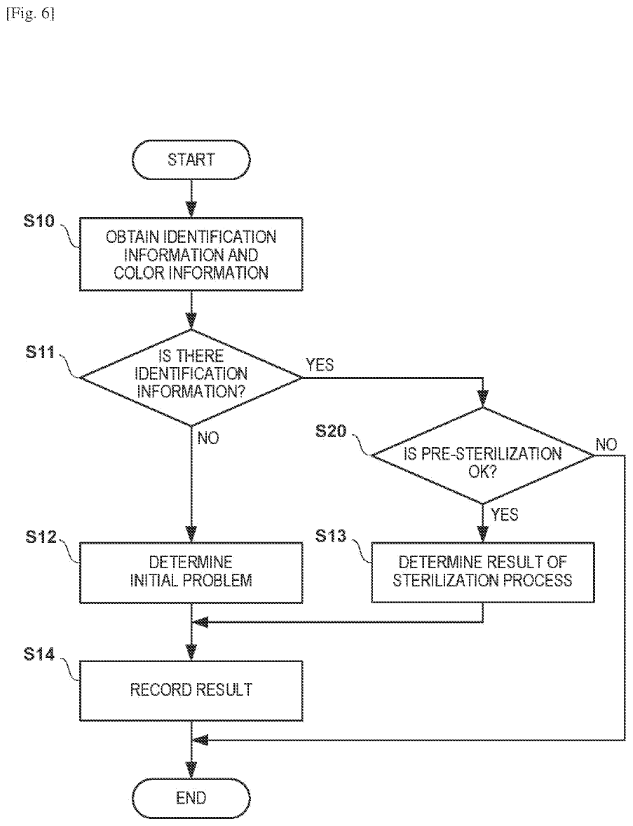

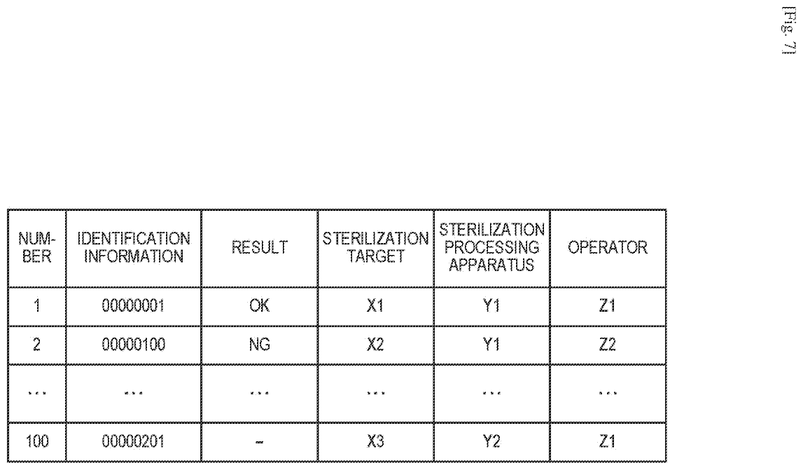

In the present embodiment, if the CI 2 has an initial problem, the PC 15 records “NG” in the pre-sterilization subfield, and displays an indication that the CI 2 cannot be used to determine the achievement degree of the sterilization process to the operator. However, it is possible that a CI 2 having an initial problem is mistakenly used in a sterilization process. FIG. 6 illustrates a revised version of the process of FIG. 5, in order to prevent this from happening. In the flowchart illustrated in FIG. 6, if a target record which corresponds to the identification information obtained from the measurement apparatus 1 is present in the management information table in step S11, the PC 15 determines whether the pre-sterilization subfield in the record for recording is “OK” in step S20. When the pre-sterilization subfield of the record for recording is “OK”, the achievement degree of the sterilization process is determined in step S13. However, if the pre-sterilization subfield of the record for recording is “NG”, the PC 15 notifies the operator that the CI 2 has an initial problem and the achievement degree of the sterilization process therefore cannot be determined. The determination of the achievement degree of the sterilization process is suspended, and the process ends. Note that if a CI 2 is used in the sterilization process without being measured by the measurement apparatus 1 before the sterilization process, the discoloration area 21 thereof normally changes from the initial color, i.e., the color value C#1. Thus with such a CI 2, the PC 15 normally determines that the CI 2 has an initial problem, and displays an indication of the initial problem to the operator. This makes it possible for the operator to recognize that it has not yet been determined whether or not the CI 2 used in the sterilization process has an initial problem.

As described thus far, a field for managing the result of determining whether or not there is an initial problem is provided in the management information table, and the initial problem determination results are managed. This configuration makes it possible to prevent a CI 2 having an initial problem from being used in a sterilization process, or, if such a CI 2 has been used, to notify the operator that such a CI 2 has been used. This in turn makes it possible to improve the quality of the sterilization process.

Although the measurement apparatus 1 and the PC 15 are separate entities in FIG. 1, the functions of the PC 15 may be incorporated into the measurement apparatus 1. In this case, the processes described as being carried out by the PC 15 are executed by the control unit 8 of the measurement apparatus 1. Furthermore, although the present embodiment describes the management apparatus 100 as including the measurement apparatus 1 and the PC 15, it is also possible for the PC 15 alone to be the management apparatus 100. Meanwhile, FIG. 1 illustrates a plurality of management apparatuses 100 being used, with the server apparatus 14 holding the management information table and the management apparatuses 100 able to access the same management information table over the network. However, in an environment such as where only a single management apparatus 100 is used, the configuration may be such that the management information table is held in the PC 15. Furthermore, instead of providing the PC 15, a configuration can be implemented in which the measurement apparatus 1 is provided with a network communication function, and the measurement apparatus 1 sends the color information and identification information to the server apparatus 14 over the network. In this case, the processing described as being carried out by the PC 15 is executed by the server apparatus 14, and the server apparatus 14 functions as the management apparatus.

### Second Embodiment

Next, a second embodiment will be described, focusing on the differences from the first embodiment. FIG. 7 illustrates the management information table according to the present embodiment. The “number” field and the “identification information” field are the same as in the first embodiment. The “result” field corresponds to the post-sterilization subfield in the first embodiment, and the sterilization process determination result based on the measured color value Ct is recorded in this field. Information specifying the sterilization target in the sterilization process is recorded in the “sterilization target” field. Information specifying the sterilization processing apparatus which sterilizes the sterilization target is stored in the “sterilization processing apparatus” field. Information specifying the operator who carries out the sterilization process is stored in the “operator” field. In the present embodiment, information is recorded into the number field, the identification information field, the sterilization target field, the sterilization processing apparatus field, and the operator field before the sterilization process is carried out, whereas information is recorded into the result field after the sterilization process is carried out. In other words, in the present embodiment, when a target record is added, the number field, the identification information field, the sterilization target field, the sterilization processing apparatus field, and the operator field are fields in which information is to be recorded. If information is already recorded in these fields when the color information and the identification information are obtained from the measurement apparatus 1, the result field is the field in which information is to be recorded.

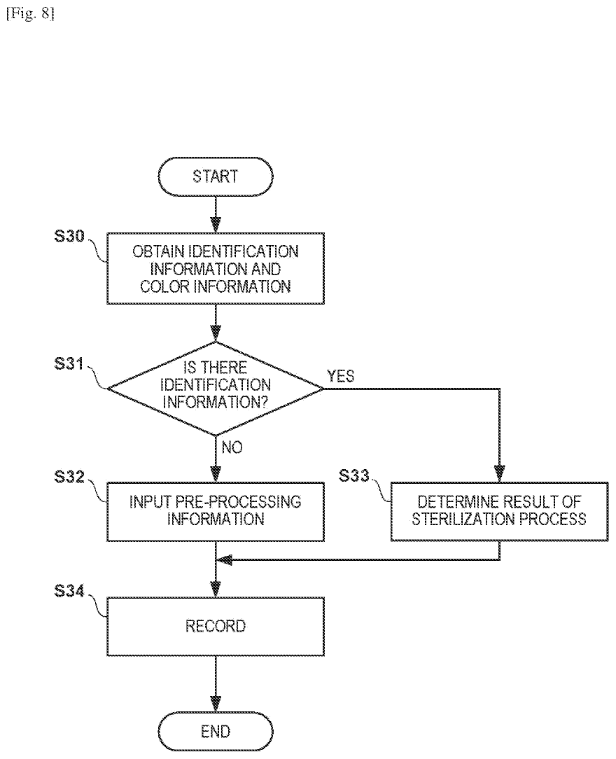

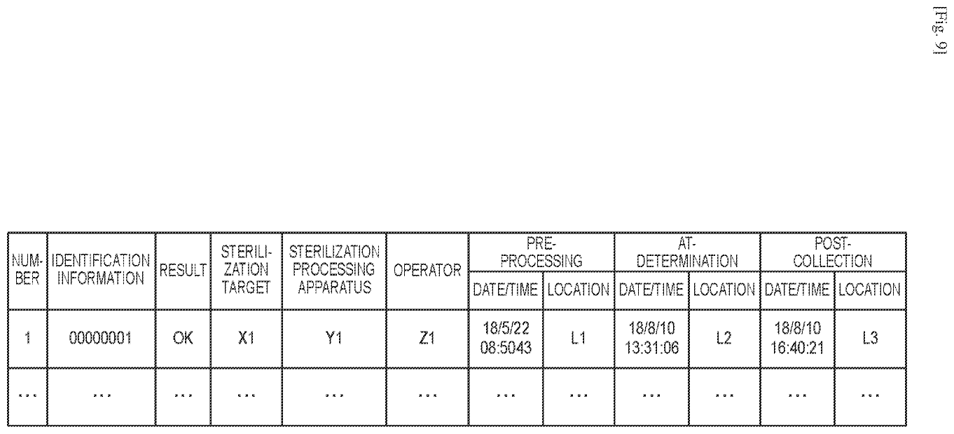

FIG. 8 is a flowchart illustrating processing carried out by the PC 15 according to the present embodiment. In step S30, the PC 15 obtains the identification information and color information from the measurement apparatus 1. Then, in step S31, the PC 15 determines whether or not a target record, in which that identification information is recorded, is present in the management information table. If the target record is not present in the management information table, the PC 15 adds a target record, in which the corresponding identification information is recorded, to the management information table. Then, in step S32, the PC 15 displays a screen prompting the operator to input related information, which is related to the sterilization process. Specifically, the PC 15 displays a screen for allowing the operator to input the information which should be recorded in the sterilization target field, the sterilization processing apparatus field, and the operator field. When the operator inputs the information to be recorded into the sterilization target field, the sterilization processing apparatus field, and the operator field in response to the screen displayed in the PC 15, the PC 15 records that information in the corresponding fields of the target record in step S34. Specifically, the assigned number is recorded in the number field of the target record, and the identification information obtained from the measurement apparatus 1 is recorded in the identification information field of the target record. The related information input by the operator is recorded in the sterilization target field, the sterilization processing apparatus field, and the operator field of the target record.

On the other hand, if in step S31 there is a target record, in which the corresponding identification information is recorded in the management information, the PC 15 determines the result of the sterilization process in step S33. Specifically, the PC 15 determines whether the sterilization process corresponds to “OK” or “NG” by comparing the color value C#1 to color value C#10 with the measured color value. The determination result is recorded in the result field in step S34.

in the present embodiment, the operator is allowed to input related information, which is required to be recorded in relation to the sterilization process for which the CI 2 is used, before the sterilization process is carried out. The information is recorded in association with the identification information of that CI 2. This clarifies the relationship between the CI 2 and an object, person, or the like related to the sterilization process carried out using that CI 2. Although the result of the sterilization process is determined immediately before using the sterilization target, e.g., immediately before surgery, recording the CI 2 and the object, person, or the like related to the sterilization process carried out using that CI 2 in advance makes it possible to prevent such information from being lost. Even if, for example, the determination result was “NG” in the measurement taken after the sterilization process, making this association in advance makes it possible to identify the sterilization processing apparatus and the like used in the sterilization process immediately and analyze the reason why the sterilization process returned “NG” with ease, on the basis of the related information that is recorded. In this manner, the operator is prompted to input the related information at the time of the measurement by the measurement apparatus 1, before the sterilization process using the CI 2, which ensures that the object, person, or the like related to the sterilization process carried out using that CI 2 can be associated reliably. Associating the CI 2 with the related information before the sterilization process makes it possible to optimize the work involved in the sterilization process and maintain a high level of quality for the sterilization process. Note that as in the first embodiment, whether the CI 2 has an initial problem can be determined in the measurement taken before the sterilization process in the present embodiment as well.

### Third Embodiment

Next, a third embodiment will be described, focusing on the differences from the second embodiment. FIG. 9 illustrates the management information table according to the present embodiment. The “number” field, the “identification information” field, the “result” field, the “sterilization target” field, the “sterilization processing apparatus” field, and the “operator” field are the same as in the second embodiment. A “preprocessing” field, an “at-determination” field, and a “post-collection” field each includes a “date/time” subfield and a “location” subfield. The date/time and location at which the CI 2 was measured by the measurement apparatus 1 before the sterilization process are recorded in the pre-processing field. Thereafter, the sterilization process is normally carried out with the sterilization target and the CI 2 held in a receptacle called a “container”. The sterilization target and the CI 2 that have been subjected to the sterilization process are stored in the container until immediately before the sterilization target is to be used. The determination of the sterilization process result is made, for example, in an operating room or the like, when the container is opened and the sterilization target and CI 2 are removed in order to use the sterilization target. The date/time and location at which the CI 2 was measured by the measurement apparatus 1 to make this determination are recorded in the at-determination field. After use, the sterilization target is collected from the location of its use, e.g., the operating room, along with the CI 2, and is then brought to a cleaning room or the like to be cleaned. The date/time and location at which the CI 2 was measured by the measurement apparatus 1 after being collected in this manner are recorded in the post-collection field.

In the present embodiment, when a target record is added, the number field, the identification information field, the sterilization target field, the sterilization processing apparatus field, the operator field, and the pre-processing field are fields in which information is to be recorded. If information is already recorded in these fields when the color information and the identification information are obtained from the measurement apparatus 1, the result field and the at-determination field are the fields in which information is to be recorded. Furthermore, if information is already recorded in the result field when the color information and the identification information are obtained from the measurement apparatus 1, the post-collection field is the field in which information is to be recorded.

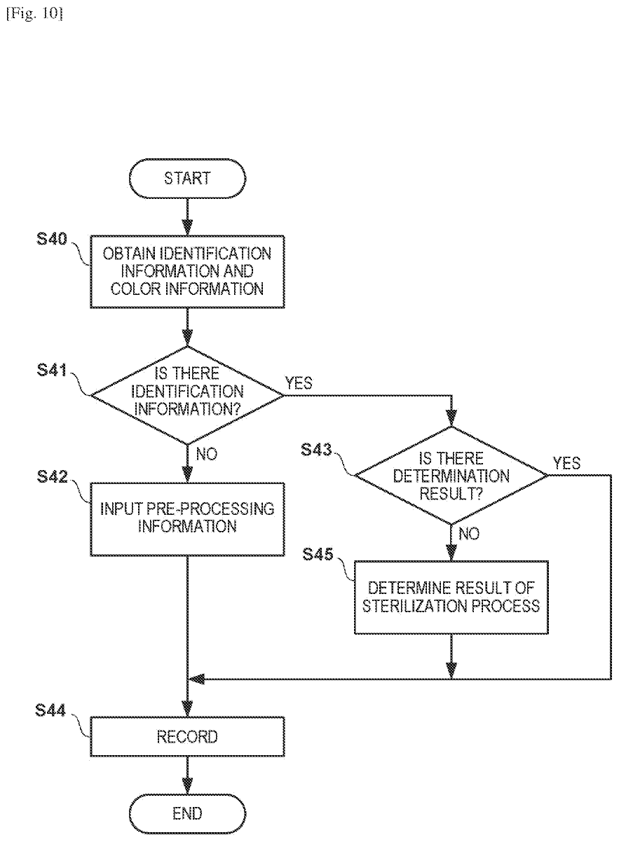

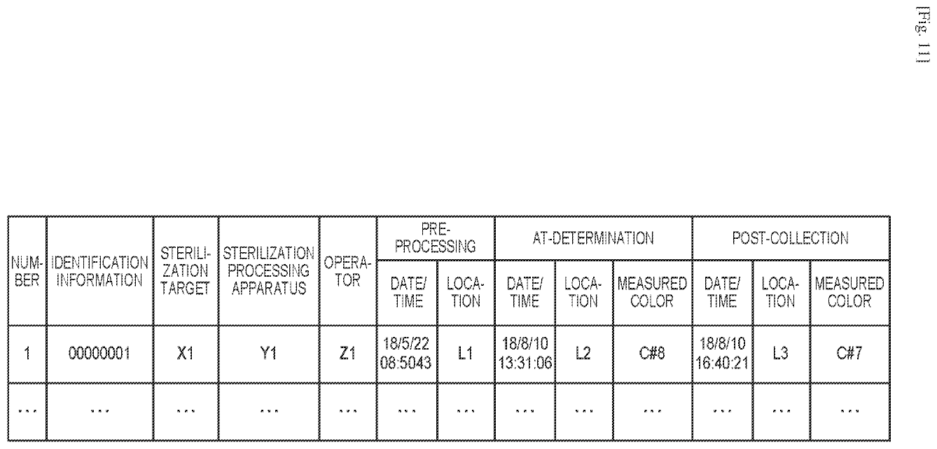

FIG. 10 is a flowchart illustrating processing carried out by the PC 15 according to the present embodiment. In step S40, the PC 15 obtains the identification information and color information from the measurement apparatus 1. Then, in step S41, the PC 15 determines whether or not a target record, in which that identification information is recorded, is present in the management information table. If the target record is not present in the management information table, the PC 15 adds a target record, in which the corresponding identification information is recorded, to the management information table. Then, in step S42, the PC 15 displays a screen allowing the operator to input related information pertaining to the sterilization process. Specifically, the PC 15 displays a screen for allowing the operator to input the information which should be recorded in the sterilization target field, the sterilization processing apparatus field, and the operator field. When the operator inputs the information to be recorded into the sterilization target field, the sterilization processing apparatus field, and the operator field in response to the screen displayed in the PC 15, the PC 15 records that information in the corresponding fields of the target record in step S44. Specifically, the assigned number is recorded in the number field of the target record, and the identification information obtained from the measurement apparatus 1 is recorded in the identification information field of the target record. The related information input by the operator is recorded in the sterilization target field, the sterilization processing apparatus field, and the operator field of the target record. Furthermore, the PC 15 records the date/time of the recording, held by the PC 15, in the date/time subfield of the pre-processing field, and records the installation location of the PC 15, which is set in that PC 15, in the location subfield of the pre-processing field.

On the other hand, if the target record is present in the management information table in step S41, the PC 15 determines, in step S43, whether or not a result is held in the result field of the target record. If no result is held, the PC 15 determines the result of the sterilization process in step S45. Specifically, the PC 15 determines whether the sterilization process corresponds to “OK” or “NG” by comparing the color value C#1 to color value C#10 with the measured color value Ct. The determination result is recorded in the result field in step S44. The PC 15 records the date/time of the recording in the date/time subfield of the at-determination field, and records the location set in that PC in the location subfield of the at-determination field. On the other hand, if a result is already held in the result field in step S43, the PC 15 records the date/time of the recording in the date/time subfield of the post-collection field, and records the location set in that PC in the location subfield of the post-collection field, in step S44.

Recording the date/time at which the measurement was taken and the location of the management apparatus 100 that took the measurement for each CI 2 measurement in this manner makes it possible to manage the current locations of individual CIs 2. Although the date/time and location are managed for three tasks in FIG. 9, if there are other tasks as well, a configuration can be implemented in which the date/time and location for those tasks are recorded as well. In other words, a configuration can be implemented in which the date/time and location are recorded for each task in a series of tasks involved in the sterilization process. Note that at the timing when the color information and the identification information have been obtained from the measurement apparatus 1, the PC 15 determines the fields to be recorded to at that timing, on the basis of the fields already recorded to in the target record.

The PC 15 has information associated with the fields to be recorded. The PC 15 can obtain this information from the server apparatus 14, for example. The PC 15 can be configured to display the information associated with the fields to be recorded, which has been determined, in a monitor. For example, a field to be recorded, which includes the pre-processing field, is associated with a character string “pre-sterilization process”. Furthermore, for example, a field to be recorded, which includes the at-determination field, is associated with a character string “sterilization process determination result”. Furthermore, a field to be recorded, which includes the post-collection field, is associated with a character string “post-collection”. As a result, if a situation arises in which the tasks being carried out by the operator do not coincide with the fields to be recorded, the operator can be made aware of that situation. In other words, even if, for example, the operator has mistakenly used a CI 2 for which the pre-sterilization process measurement has not yet been taken in the sterilization process, the operator can recognize his or her mistake by checking the details displayed in the monitor, and can then redo the task.

Additionally, the CI 2 is measured when, for example, the CI 2 is delivered, and the date/time is recorded in an additional field not present in FIG. 9. Then, the measurement of the pre-processing CI 2 is carried out immediately before the sterilization process. In this case, comparing the two dates/times makes it possible to determine how long has passed following the delivery. If a long time has passed following the delivery of a CI 2, the discoloration area 21 may have changed. Therefore, a configuration can be implemented in which, for example, when the amount of time that has passed following the delivery of the CI 2 is longer than a threshold, an indication to that effect is displayed to the operator to prompt him or her to not use that CI 2 in the next sterilization process. Additionally, as described above, the sterilization target and the CI 2 are kept inside a container after the sterilization process. The sterilization target is removed from the container immediately before use, and the result of the sterilization process is determined at that time by measuring the CI 2. However, the discoloration area 21 of a CI 2 which has been kept inside a container for a long period of time may have changed immediately after the sterilization process. This can be a cause of erroneous determinations for the results of sterilization processes. Furthermore, the sterilization state of a sterilization target which has been kept inside a container for a long time may not be the same as the state immediately after the sterilization process, due to the container being exposed to outside air for a long period of time. This, too, can be a cause of erroneous determinations for the results of sterilization processes, regardless of changes in the discoloration area 21 of the CI 2. A configuration can therefore be implemented in which the time within the container is determined when determining the result of the sterilization process, on the basis of the date/time recorded in the date/time subfield of the pre-processing field and the date/time recorded in the date/time subfield of the at-determination field. In this configuration, if the time within the container is longer than a predetermined amount of time, management apparatus 100 can display, to the operator or the like, an indication that the CI 2 has been kept in the container for longer than the predetermined amount of time.

In FIG. 9, “OK” or “NG” is recorded in the result field after the sterilization process, and only the date/time and location are recorded during other measurements. However, as illustrated in FIG. 11, a configuration can also be implemented in which after the sterilization process, the achievement degree, which is based on the color of the discoloration area 21, is recorded each time the CI 2 is measured. In FIG. 11, the result field in FIG. 9 has been removed, and instead, a measured color subfield has been added to the at-determination field and the post-collection field. The achievement degree determined for the discoloration area 21 is recorded in the measured color subfield. Note that in FIG. 11, the difference between the achievement degree in the at-determination field and the completeness in the post-collection field has resulted from changes over time. Whether the sterilization process is “OK” or “NG” is determined on the basis of the measured color subfield in the at-determination field.

As described thus far, timings at which to measure the CI 2 in the series of tasks involved in the sterilization process is determined in advance. The date/time, location, and the like at which the task is carried out at each timing are then recorded. This makes it possible to quickly search out the current location, state, and so on of each CI 2, and quickly confirm the time each CI 2 has been kept and so on. Managing the records pertaining to the sterilization process in this manner makes it possible to prevent human error. The series of tasks involved in the sterilization process can also be verified more easily, which makes it possible to improve the quality of the tasks. Note that as in the first embodiment, whether the CI 2 has an initial problem can be determined in the measurement taken before the sterilization process in the present embodiment as well.

Function Blocks of PC 15

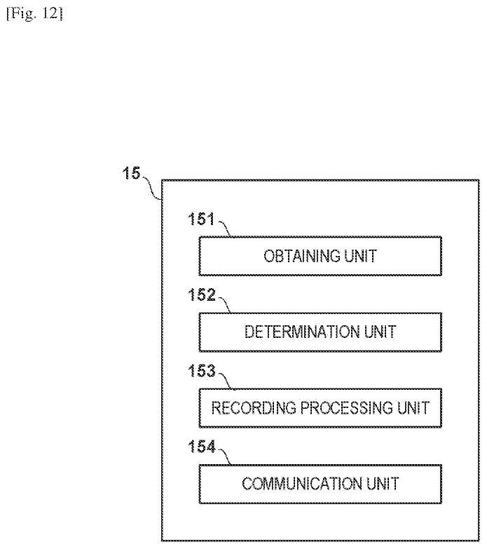

The PC 15 is a generic computer including one or more processors, volatile memory, and non-volatile memory, and carries out the above-described processes by executing programs using the one or more processors. FIG. 12 is a function block diagram illustrating the functions of the PC 15 realized by executing the programs, An obtaining unit 151 obtains the color information and the identification information from the measurement apparatus 1. Note that the PC 15 and the measurement apparatus 1 are connected by a hard-wired or wireless connection. A communication unit 154 communicates with the server apparatus 14 over a network. A recording processing unit 153 accesses the server apparatus 14 through the communication unit 154 and manipulates the management information table held by the server apparatus 14, “Manipulating” the management information table includes adding records and recording information into the fields of those records. A determination unit 152 determines an initial problem in the CI 2, the achievement degree of a sterilization process carried out using the CI 2, and the like on the basis of the color information obtained by the obtaining unit. The recording processing unit 153 determines whether or not a target record is present in the management information table, and if no such record is present, carries out a process for adding a target record to the management information table. Furthermore, on the basis of fields in the target record which have already been recorded to, the recording processing unit 153 determines the fields to be recorded in the target record, and carries out a process for recording information into the fields to be recorded which have been determined. Note that the present invention is not limited to a form in which the function blocks illustrated in FIG. 12 are realized by a generic computer. Some or all of the function blocks illustrated in FIG. 12 can be realized by any desired dedicated hardware.

Regarding the CI

As illustrated in FIG. 3, in the above embodiments, the CI 2 has a rectangular discoloration area 21, which is of a color different from the base. The color of the discoloration area 21 changes in accordance with the achievement degree of the sterilization process. The “color” of the discoloration area 21 also includes a transparent state. That is, even if the discoloration area 21 is initially transparent, and a color appears in accordance with the achievement degree of the sterilization process, that situation is included in the concept of “discoloration” or “color changing” of the dis- coloration area 21 according to the present invention. Likewise, even if the discoloration area 21 is not transparent initially, but the discoloration area 21 becomes transparent in accordance with the completeness of the sterilization process, that situation is included in the concept of “discoloration” or “color changing” of the discoloration area 21 according to the present invention. The base part may be transparent as well.

Additionally, the color of the discoloration area 21 may initially be the same as the color of the base, for example. Furthermore, the entire discoloration area 21 need not change color uniformly in accordance with the achievement degree of the sterilization process. For example, even if the entire discoloration area 21 is initially the same color as the base, but a lengthwise part thereof changes to a color different from that of the base in accordance with the achievement degree of the sterilization process, that situation is included in the concept of “discoloration” or “color changing” of the discoloration area 21 according to the present invention. Note that the discoloration area 21 may have a part that is a different color from the base initially, with the part having a different color from the base growing longer in accordance with the achievement degree of the sterilization process. Conversely, the part having a different color from the base may grow shorter in accordance with the achievement degree of the sterilization process. With such a CI 2, the achievement degree of the sterilization process is determined from the part in which the color of the discoloration area 21 has changed, or the length of the part in which the color has not changed.

Other Embodiments

The present invention can be realized as the management system illustrated in FIG. 1. The present invention can furthermore be realized as a management method, described with reference to the respective flowcharts.

Embodiments of the present invention can also be realized by a computer of a system or apparatus that reads out and executes computer executable instructions (e.g., one or more programs) recorded on a storage medium (which may also be referred to more fully as ‘non-transitory computer-readable storage medium’) to perform the functions of one or more of the above-described embodiments and/or that includes one or more circuits (e.g., application specific integrated circuit (ASIC)) for performing the functions of one or more of the above-described embodiments, and by a method performed by the computer of the system or apparatus by, for example, reading out and executing the computer executable instructions from the storage medium to perform the functions of one or more of the above-described embodiments and/or controlling the one or more circuits to perform the functions of one or more of the above-described embodiments. The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments, The scope of the following claims is to be accorded the broadest interpretation so as to encompass all such modifications and equivalent structures and functions.

This application claims the benefit of Japanese Patent Application No. 2018-222689, filed on Nov. 28, 2018, which is hereby incorporated by reference herein in its entirety.

1. A management apparatus that manages a record of a predetermined process carried out using test paper, the test paper having identification information and a discoloration area, a color of the discoloration area changing in accordance with an achievement degree of the predetermined process, the apparatus comprising:
one or more processors; and
one or more memories including a program,
wherein the program, when executed by the one or more processors, causes the one or more processors to perform operations including:
obtaining the identification information and a measurement result for the discoloration area, from a measurement apparatus that measures the discoloration area of the test paper and reads the identification information;
determining a field to be recorded in a target record based on already-recorded fields in the target record which, among records recording the predetermined process, corresponds to the identification information; and
recording information in the determined field to be recorded,
wherein the record includes a first field in which the achievement degree of the predetermined process is recorded, and one or more second fields in which information is recorded earlier than a timing at which the achievement degree of the predetermined process is recorded in the first field.

2. The management apparatus according to claim 1,
wherein the operations further include displaying information associated with the determined field to be recorded.

3. The management apparatus according to claim 1, wherein the operations further include:
determining the achievement degree of the predetermined process based on the measurement result for the discoloration area obtained, at a timing at which the first field is included in the field to be recorded, and
recording the achievement degree of the predetermined process, in the first field of the target record.

4. The management apparatus according to claim 3,
wherein the one or more second fields include a third field in which a determination result of an initial problem in the test paper is recorded; and
the operations further include determining the initial problem of the test paper based on the measurement result for the discoloration area obtained at a timing at which the third field is included in the field to be recorded.

5. The management apparatus according to claim 4,
wherein the operations further include determining the initial problem of the test paper by comparing the measurement result for the discoloration area with a predetermined initial color of the discoloration area.

6. The management apparatus according to claim 4,
wherein the operations further include, in response to determining that the test paper has the initial problem, displaying that the test paper has the initial problem.

7. The management apparatus according to claim 4,
wherein the operations further include recording the determination result of the initial problem in the test paper in the third field of the target record.

8. The management apparatus according to claim 7,
wherein the operations further include determining whether or not the determination result recorded in the third field indicates the initial problem in the test paper at the timing at which the first field is included in the field to be recorded.

9. The management apparatus according to claim 8,
wherein the operations further include suspending the determination of the achievement degree of the predetermined process when the determination result recorded in the third field indicates the initial problem in the test paper.

10. The management apparatus according to claim 8,
wherein the operations further include, in a case where the determination result recorded in the third field indicates that the test paper has the initial problem, displaying that the test paper has an initial problem.

11. The management apparatus according to claim 1,
wherein the one or more second fields include a fourth field in which at least one of a target of the predetermined process, an apparatus used in the predetermined process, and an operator of the predetermined process is recorded.

12. The management apparatus according to claim 11,
wherein the operations further include, in a case where the fourth field is included in the field to be recorded, prompting an input of information to be recorded in the fourth field.

13. The management apparatus according to claim 1,
wherein each field to be recorded includes a field in which a date/time is recorded.

14. The management apparatus according to claim 1,
wherein each field to be recorded includes a field in which a location is recorded.

15. The management apparatus according to claim 1,
wherein the operations further include, in a case where the target record is not present in a management information table, adding the target record to the management information table.

16. The management apparatus according to claim 15, wherein the management apparatus holds the management information table.

17. The management apparatus according to claim 15, wherein the operations further include communicating with an external apparatus that holds the management information table to obtain information recorded in the management information table and communicate information to be recorded in the management information table to the external apparatus.

18. The management apparatus according to claim 1,
wherein the management apparatus includes the measurement apparatus.

19. The management apparatus according to claim 18,
wherein the measurement apparatus reads the identification information, the identification information being printed on the test paper.

20. The management apparatus according to claim 18,
wherein the measurement apparatus reads the identification information by communicating with a radio tag provided on the test paper.

21. The management apparatus according to claim 1,
wherein the predetermined process is a sterilization process.

22. A non-transitory computer readable storage medium including a program that, when executed by one or more processors in an apparatus including the one or more processors, causes the apparatus to function as a management apparatus that manages a record of a predetermined process carried out using test paper, the test paper having identification information and a discoloration area, a color of the discoloration area changing in accordance with an achievement degree of the predetermined process, and to perform operations including:
obtaining the identification information and a measurement result for the discoloration area, from a measurement apparatus that measures the discoloration area of the test paper and reads the identification information;
determining a field to be recorded in a target record based on already-recorded fields in the target record which, among records recording the predetermined process, corresponds to the identification information; and
recording information in the determined field to be recorded,
wherein the record includes a first field in which the achievement degree of the predetermined process is recorded, and one or more second fields in which information is recorded earlier than a timing at which the achievement degree of the predetermined process is recorded in the first field.

23. A management system that manages a record of a predetermined process carried out using test paper, the test paper having identification information and a discoloration area, a color of the discoloration area changing in accordance with an achievement degree of the predetermined process, the system comprising:
a holding unit configured to hold a record, corresponding to identification information, in which the predetermined process is recorded; and
a recording unit configured to record information in the record,
wherein the record includes a field in which information is recorded before the test paper having the identification information corresponding to that record is used in the predetermined process, and a field in which information is recorded after the test paper having the identification information corresponding to that record has been used in the predetermined process.

24. The management system according to claim 23,
wherein the field in which information is recorded after the test paper has been used in the predetermined process includes a first field in which the achievement degree of the predetermined process is recorded.

25. The management system according to claim 24, further comprising:
a measuring unit configured to measure the discoloration area of the test paper; and
a determining unit configured to determine the achievement degree of the predetermined process to be recorded in the first field based on a measurement result of the discoloration area by the measuring unit.

26. The management system according to claim 24,
wherein the field in which information is recorded before the test paper is used in the predetermined process includes a second field in which a determination result of an initial problem in the test paper is recorded.

27. The management system according to claim 26, further comprising:
a measuring unit configured to measure the discoloration area of the test paper; and
a determining unit configured to determine the determination result of the initial problem in the test paper, to be recorded in the second field, based on a measurement result of the discoloration area by the measuring unit.

28. The management system according to claim 2,
wherein the field in which information is recorded before the test paper is used in the predetermined process includes a third field in which at least one of a target of the predetermined process, an apparatus used in the predetermined process, and an operator of the predetermined process is recorded.

29. A management method that manages a record of a predetermined process carried out using test paper, the test paper having identification information and a discoloration area, a color of the discoloration area changing in accordance with an achievement degree of the predetermined process, the method comprising:
reading identification information of the test paper; and
determining a field to be recorded in a target record based on already-recorded fields in the target record which, among records recording the predetermined process, corresponds to the read identification information, and recording information in the determined field to be recorded,
wherein the record includes a first field in which the achievement degree of the predetermined process, based on a measurement result of the discoloration area, is recorded, and one or more second fields in which information is recorded earlier than a timing at which the achievement degree of the predetermined process is recorded in the first field.

30. The management method according to claim 29,
wherein the one or more second fields includes at least one of a field in which a determination result an initial problem in the test paper is recorded, a field in which a target of the predetermined process is recorded, a field in which an apparatus used in the predetermined process is recorded, and a field in which an operator of the predetermined process is recorded.
